(19) United States
(12) Patent Application Publication
(10) Pub. No.: US 2016/0184600 A1
(43) Pub. Date: Jun. 30, 2016

(54) MAGNETIC EXCITATION DEVICE AND ELECTROMAGNETIC THERMAL ABLATION APPARATUS CONTAINING THE SAME

(71) Applicant: Metal Industries Research and Development Centre, Kaohsiung (TW)

(72) Inventors: Tung-Chieh Yang, Kaohsiung (TW); Ming-Hui Chen, Kaohsiung (TW); Yu-Fen Kuo, Kaohsiung (TW); Yan-Jun Chen, Kaohsiung (TW); Yu-Min Ting, Kaohsiung (TW); Tsung-Chih Yu, Kaohsiung (TW); Ho-Chung Fu, Kaohsiung (TW)

(21) Appl. No.: 14/586,019

(22) Filed: Dec. 30, 2014

(51) Int. Cl. A61N 2/02; A61B 18/04; A61N 1/40

(52) U.S. Cl. CPC A61N 2/02; A61N 1/403; A61B 18/04; A61B 2018/00577

(57) ABSTRACT

A magnetic excitation device for an electromagnetic thermal ablation apparatus includes a magnetic unit and two coil units. The magnetic unit has two magnetic induction ends that are spaced apart from each other. The coil units are respectively disposed around the magnetic induction ends. Each of the coil units has two connection parts connectible to an alternating electric power source for generating an alternating magnetic field.

### BACKGROUND OF THE INVENTION

1. Field of the Invention

This invention relates to a magnetic excitation device, more particularly to a magnetic excitation device for treating a tumor. This invention also relates to an electromagnetic thermal ablation apparatus which is used for treating a tumor and which includes the magnetic excitation device.

2. Description of the Related Art

Conventional methods for treating a tumor include, for example, microwave thermal ablation and radiofrequency thermal ablation which cause local tumor tissues to necrotize so as to achieve the purpose of treating the tumor. However, such conventional treating methods are costly and cause patients considerable pain and are thus unacceptable for most patients.

Another method developed for treating a tumor is magnetic thermal ablation, which uses a magnetic induction needle effected by an alternating magnetic field to produce eddy current which is then converted to thermal energy for thermal ablation treatment of the tumor tissues. The cost and the pain the patient has to endure may be reduced significantly by this treatment method. However, there is still a need for modifying the control of the magnetic field for this treatment method so as to enhance the treatment quality.

Referring to FIG. 1, an existing electromagnetic thermal ablation apparatus is shown to include a machine table 11, an electromagnetic controlling device 12 disposed on the machine table 11, a coil 13 electrically connected to the electromagnetic controlling device 12, a cooling water circulation device 14 connected to the electromagnetic controlling device 12, and a central control device 15 electrically connected to the electromagnetic controlling device 12 and to the cooling water circulation device 14. With regard to a superficial electromagnetic ablation, the coil 13 is disposed above a body of a patient, and a magnetic induction needle (not shown) is passed through a high-frequency magnetic field produced after the coil 13 is electrified so that the magnetic induction needle produces thermal energy to ablate the tumor tissues. With regard to a deep electromagnetic ablation, two of the electromagnetic thermal ablation apparatuses are required. The coils 13 of these two electromagnetic thermal ablation apparatuses are respectively disposed above and below the tumor tissues of the patient to be treated. The coils 13 produce the high-frequency magnetic field after the electromagnetic controlling devices 12 of both of the electromagnetic thermal ablation apparatuses are electrified so that the magnetic induction needle produces thermal energy to ablate the tumor tissues.

The coil 13 of the existing electromagnetic thermal ablation apparatus cannot be positioned easily and the elevation level thereof and/or the distance between two of the electromagnetic thermal ablation apparatuses can only be adjusted manually. Usually, the horizontal angle of the coil 13 of the existing electromagnetic thermal ablation apparatus cannot be adjusted. Therefore, the adjustment flexibility of the coil 13 is limited.

### SUMMARY OF THE INVENTION

Therefore, an object of the present invention is to provide a magnetic excitation device which can produce a concentrated and deep magnetic field.

Another object of the present invention is to provide an electromagnetic thermal ablation apparatus which can automatically and precisely control the position and the angle of the magnetic excitation device so that the magnetic excitation device can produce a concentrated and deep magnetic field so as to enhance the treatment effect.

According to one aspect of this invention, there is provided a magnetic excitation device for an electromagnetic thermal ablation apparatus, which includes a magnetic unit and two coil units. The magnetic unit has two magnetic induction ends that are spaced apart from each other. The coil units are respectively disposed around the magnetic induction ends. Each of the coil units has two connection parts connectible to an alternating electric power source for generating an alternating magnetic field.

According to another aspect of this invention, there is provided an electromagnetic thermal ablation apparatus, which includes a carrier unit, a universal rotation unit, a cantilever unit, a magnetic excitation device and a control unit. The carrier unit includes a carrier body, and a plurality of wheels disposed at a bottom of the carrier body. The universal rotation unit is connected pivotally to the carrier unit and is rotatable to different angles relative to the carrier unit. The cantilever unit is connected pivotally to the universal rotation unit. The magnetic excitation device is connected pivotally to the cantilever unit and is opposite to the universal rotation unit, and includes a magnetic unit and two coil units. The magnetic unit has two magnetic induction ends that are spaced apart from each other. The coil units are respectively disposed around the magnetic induction ends. Each of the coil units has two connection parts connectible to an alternating electric power source for generating an alternating magnetic field. The control unit is disposed on the carrier unit and is settable to control rotation of the universal rotation unit that brings the cantilever arm and the magnetic excitation device to move.

### BRIEF DESCRIPTION OF THE DRAWINGS

Other features and advantages of the present invention will become apparent in the following detailed description of the embodiments with reference to the accompanying drawings, of which:

FIG. 1 is a perspective view of an existing electromagnetic thermal ablation apparatus;

FIG. 2 is a schematic view of a first embodiment of a magnetic excitation device according to the present invention;

FIG. 3 is a schematic view of a second embodiment of a magnetic excitation device according to the present invention;

FIG. 4 is a schematic view of a first embodiment of an electromagnetic thermal ablation apparatus according to the present invention;

FIG. 5 is another schematic view of the first embodiment of the electromagnetic thermal ablation apparatus according to the present invention; and

FIG. 6 is a perspective view of a second embodiment of the electromagnetic thermal ablation apparatus according to the present invention in a use state.

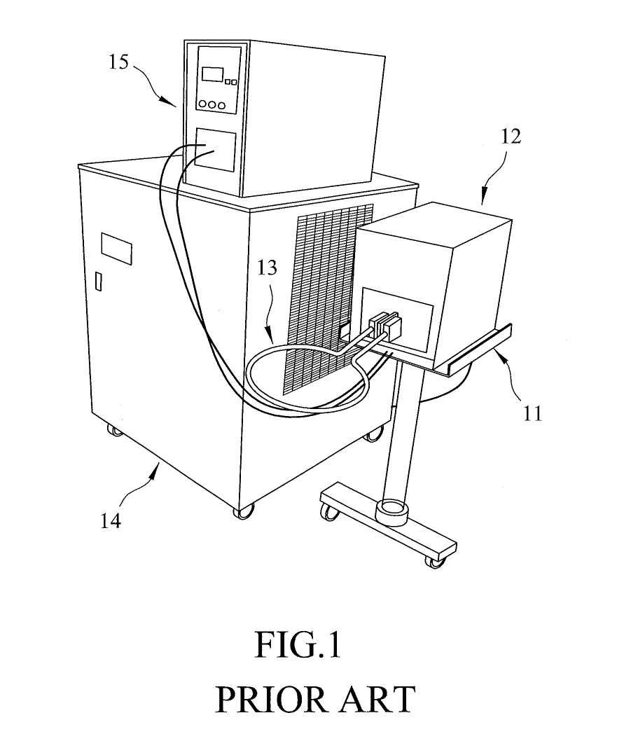

### DETAILED DESCRIPTION OF AN ILLUSTRATIVE EMBODIMENT

Before the present invention is described in greater detail with reference to the accompanying embodiments, it should be noted herein that like elements are denoted by the same reference numerals throughout the disclosure.

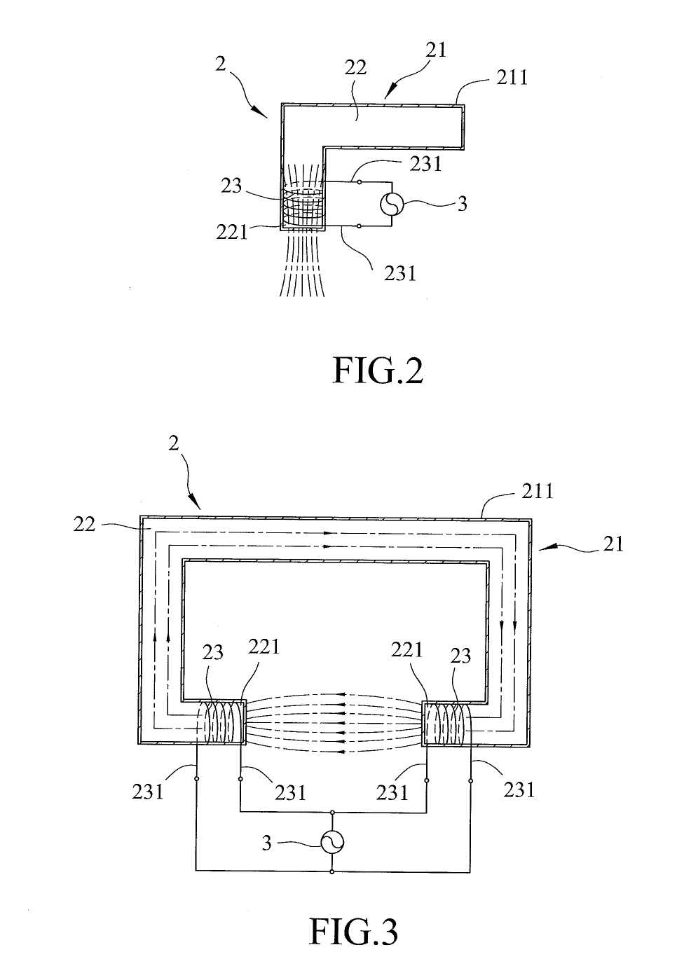

Referring to FIG. 2, the first embodiment of a magnetic excitation device 2 according to the present invention includes an insulating shell body 21, a magnetic unit 22 and a coil unit 23.

The insulating shell body 21 covers the magnetic unit 22 and is made of a heat-insulating, non-magnetizable, non-conductive and non-magnetic-field-shielding material. The insulating shell body 22 has two shell halves 211 that are separably mated with each other. Only one of the shell halves 211 is shown in FIG. 2.

The magnetic unit 22 is made of a material selected from the group consisting of a highly magnetic monocrystalline material, a highly magnetic polycrystalline material and a ferromagnetic material (for example, Mn—Zn ferrite, Ni—Zn ferrite). The magnetic unit 22 has, for example, an L-shape and has a magnetic induction end 221.

The coil unit 23 is disposed around the magnetic induction end 221 of the magnetic unit 22. The coil unit 23 has two connection parts 231 connectible to an alternating electric power source 3 for generating an alternating magnetic field. The coil unit 23 may be wound in a manner of single-layer close winding, equal-pitch winding, multi-layer close winding, arbitrary winding, alignment winding, positioning winding, parallel winding and the like.

The magnetic excitation device 2 is useful in an electromagnetic thermal ablation apparatus. The coil unit 23 connected to the alternating electric power source 3 via the connection parts 231 may generate a concentrated and deep magnetic field. When the magnetic excitation device 2 is disposed close to or above a skin of a human body, an end portion of an auxiliary treatment tool (for example, a magnetic induction needle) is passed through the magnetic field and is then punctured into the human body to reach the tumor tissues to be treated. An eddy current is produced at the end portion of the auxiliary treatment tool under the action of the magnetic field and is then converted to thermal energy to conduct a deep thermal ablation of the tumor tissues.

Referring to FIG. 3, the second embodiment of a magnetic excitation device 2 according to the present invention includes an insulating shell body 21, a magnetic unit 22 and two coil units 23.

The magnetic unit 22 has two magnetic induction ends 221 that are spaced apart from each other. The magnetic unit 22 has a shape selected from the group consisting of a C-shape, an inverted U-shape and a U-shape. As described above, the magnetic unit 22 is made of a material selected from the group consisting of a highly magnetic monocrystalline material, a highly magnetic polycrystalline material and a ferromagnetic material.

The coil units 23 are respectively disposed around the magnetic induction ends 221. Each of the coil units 23 has two connection parts 231 connectible to an alternating electric power source 3 for generating an alternating magnetic field.

As describe above, the insulating shell body 21 covers the magnetic unit 22 and is made of a heat-insulating, non-magnetizable, non-conductive and non-magnetic-field-shielding material. The insulating shell body 22 has two shell halves 211 that are separably mated with each other. Only one of the shell halves 211 is shown in FIG. 3.

When the second embodiment of the magnetic excitation device 2 is used in an electromagnetic thermal ablation apparatus, the magnetic induction ends 221 of the magnetic excitation device 2 are respectively disposed above and below a skin of a human body. The coil units 23 connected to the alternating electric power source 3 via the connection parts 231 may generate a concentrated and deep magnetic field. An end portion of an auxiliary treatment tool (for example, a magnetic induction needle) is passed through the magnetic field and is then punctured into the human body to reach the tumor tissues to be treated. An eddy current is produced at the end portion of the auxiliary treatment tool under the action of the magnetic field and is then converted to thermal energy to conduct a deep thermal ablation of the tumor tissues.

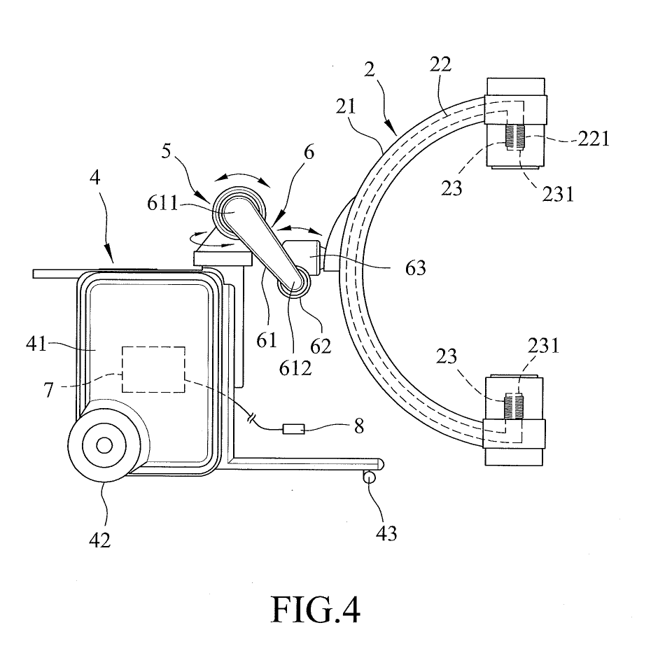

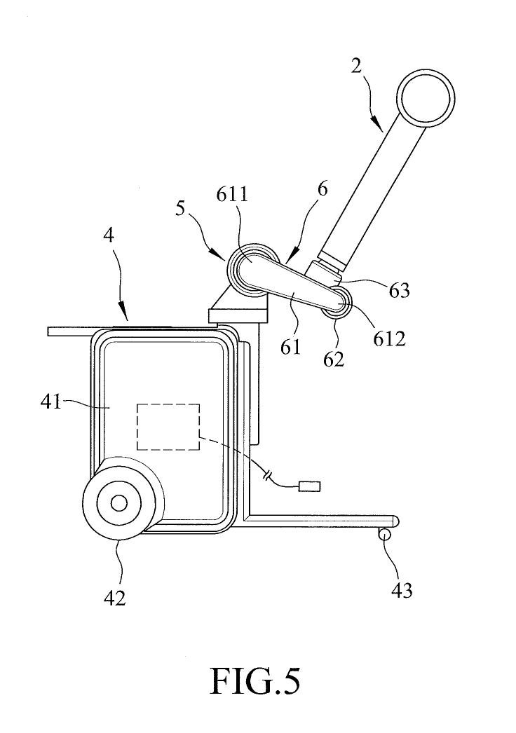

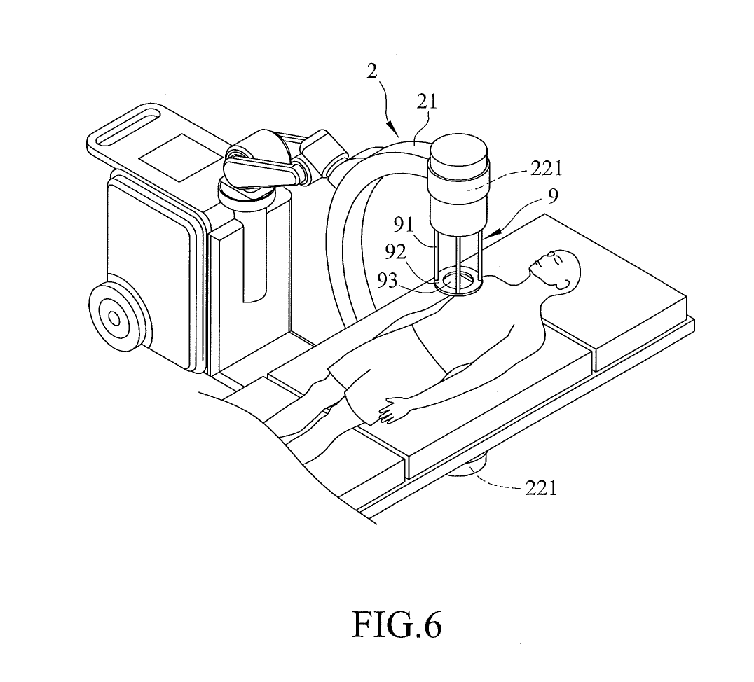

Referring to FIGS. 4 and 5, a first embodiment of an electromagnetic thermal ablation apparatus according to the present invention includes a carrier unit 4, a universal rotation unit 5, a cantilever unit 6, a magnetic excitation device 2, a control unit 7 and an input unit 8.

The carrier unit 4 includes a carrier body 41, and a plurality of wheels 42, 43 disposed at a bottom of the carrier body 41.

The universal rotation unit 5 is connected pivotally to the carrier unit 4 and is rotatable to different angles ranging from 0 to 360 degrees relative to the carrier unit 4.

The cantilever unit 6 is connected pivotally to the universal rotation unit 5, and includes two cantilever arms 61 (only one of the cantilever arms 61 is shown?), an interconnecting lever 62 interconnecting the cantilever arms 61, and a rotating shaft 63 connected pivotally to the interconnecting lever 62. Specifically, each of the cantilever arms 61 has a first end portion 611 and a second end portion 612 opposite to the first end portion 611. The first end portions 611 of the cantilever arms 61 are connected pivotally to two sides of the universal rotation unit 5 correspondingly. The interconnecting lever 62 interconnects the second end portions 612 of the cantilever arms 61.

The magnetic excitation device 2 is connected to the rotating shaft 63 of the cantilever unit 6 and is opposite to the universal rotation unit 5 so that the magnetic excitation device 2 is rotatable relative to the rotating shaft 63 of the cantilever unit 6. Therefore, the magnetic excitation device 2 may be rotated flexibly via the aforesaid pivotal connection mechanism.

The aforesaid second embodiment of the magnetic excitation device 2 is used in the first embodiment of the electromagnetic thermal ablation apparatus.

The control unit 7 is disposed on the carrier unit 4 and is settable to control rotation of the universal rotation unit 5 that brings the cantilever unit 6 and the magnetic excitation device 2 to move.

The input unit 8 manually inputs and transmits wirelessly or non-wirelessly a control signal to the control unit 7 such that the control unit 7 is able to control rotation of the universal rotation unit 5 together with the cantilever unit 6 and the magnetic excitation device 2. It is noted that the connection parts 231 of the coil units 23 of the magnetic excitation device 2 are electrically connected to the control unit 7.

In use of the electromagnetic thermal ablation apparatus of the present invention, the control instruction is input via the input unit 8 and the signal of the control instruction is transmitted to the control unit 7 so as to control rotation of the universal rotation unit 5 that brings the cantilever unit 6 and the magnetic excitation device 2 to move so as to position the magnetic excitation device 2 in a desirable place in which the magnetic induction ends 221 of the magnetic excitation device 2 are respectively disposed above and below a skin of a human body. The coil units 23 connected to the alternating electric power source 3 (see FIG. 3) may generate a concentrated and deep magnetic field. An end portion of an auxiliary treatment tool (for example, a magnetic induction needle) is passed through the magnetic field and is then punctured into the human body to reach the tumor tissues to be treated. An eddy current is produced at the end portion of the is auxiliary treatment tool under the action of the magnetic field and is then converted to thermal energy to conduct a deep thermal ablation of the tumor tissues.

Referring to FIG. 6, a second embodiment of an electromagnetic thermal ablation apparatus according to the present invention is similar to the first embodiment of the electromagnetic thermal ablation apparatus except that the second embodiment of the electromagnetic thermal ablation apparatus further includes an auxiliary magnetic unit 9. The auxiliary magnetic unit 9 includes a mounting bracket 91 connected to one end of the insulating shell body 21, and a base member 92 made of a magnetic material and attached to the mounting bracket 91. The base member 92 has an opening 93. The depth and strength of the magnetic field produced by the magnetic excitation device 2 may be enhanced by the auxiliary magnetic unit 9 so as to further raise the treatment effect.

While the present invention has been described in connection with what are considered the most practical embodiments, it is understood that this invention is not limited to the disclosed embodiments but is intended to cover various arrangements included within the spirit and scope of the broadest interpretation and equivalent arrangements.

1. A magnetic excitation device for an electromagnetic thermal ablation apparatus, comprising:
a magnetic unit having two magnetic induction ends that are spaced apart from each other; and
two coil units that are respectively disposed around said magnetic induction ends, each of said coil units having two connection parts connectible to an alternating electric power source for generating an alternating magnetic field.

2. The magnetic excitation device according to claim 1, wherein said magnetic unit is made of a material selected from the group consisting of a highly magnetic monocrystalline material, a highly magnetic polycrystalline material and a ferromagnetic material, said magnetic unit having a shape selected from the group consisting of a C-shape, an inverted U-shape and a U-shape.

3. The magnetic excitation device according to claim 1, further comprising an insulating shell body that covers said magnetic unit and that is made of a heat-insulating, non-magnetizable, non-conductive and non-magnetic-field-shielding material, said insulating shell body having two shell halves that are separably mated with each other.

4. An electromagnetic thermal ablation apparatus, comprising:
a carrier unit including a carrier body, and a plurality of wheels disposed at a bottom of said carrier body;
a universal rotation unit connected pivotally to said carrier unit and rotatable to different angles relative to said carrier unit;
a cantilever unit connected pivotally to said universal rotation unit;
a magnetic excitation device connected to said cantilever unit and being opposite to said universal rotation unit, and including
a magnetic unit having two magnetic induction ends that are spaced apart from each other, and
two coil units that are respectively disposed around said magnetic induction ends, each of said coil units having two connection parts connectible to an alternating electric power source for generating an alternating magnetic field; and
a control unit disposed on said carrier unit and settable to control rotation of said universal rotation unit that brings said cantilever unit and said magnetic excitation device to move.

5. The electromagnetic thermal ablation apparatus according to claim 4, wherein said magnetic unit is made of a material selected from the group consisting of a highly magnetic monocrystalline material, a highly magnetic polycrystalline material and a ferromagnetic material, said magnetic unit having a shape selected from the group consisting of a C-shape, an inverted U-shape and a U-shape.

6. The electromagnetic thermal ablation apparatus according to claim 4, further comprising an input unit to manually input and to transmit wirelessly or non-wirelessly a control signal to said control unit such that said control unit is able to control rotation of said universal rotation unit together with said cantilever unit and said magnetic excitation device.

7. The electromagnetic thermal ablation apparatus according to claim 4, wherein said magnetic excitation device further includes an insulating shell body that covers said magnetic unit and that is made of a heat-insulating, non-magnetizable, non-conductive and non-magnetic-field-shielding material, said insulating shell body having two shell halves that are separably mated with each other.

8. The electromagnetic thermal ablation apparatus according to claim 7, further comprising an auxiliary magnetic unit that includes a mounting bracket connected to one end of said insulating shell body, and a base member made of a magnetic material and attached to said mounting bracket, said base member having an opening.
